(19) United States
(12) Patent Application Publication
(10) Pub. No.: US 2026/0245470 A1
(43) Pub. Date: Aug. 20, 2026

(54) INTELLIGENT MEDICAL COMMUNICATION INTERACTION TRAINING SYSTEM BASED ON VIRTUAL CHARACTERS

(71) Applicant: TAIPEI MEDICAL UNIVERSITY, Taipei City (TW)

(72) Inventor: Jen-Chieh WU, Taipei City (TW)

(21) Appl. No.: 19/250,275

(22) Filed: Jun. 26, 2025

(30) Foreign Application Priority Data
Feb. 19, 2025 (TW) 114106119

(51) Int. Cl. G09B 5/06; G06T 11/60; G06V 40/16; G10L 15/18; G10L 15/30; G10L 25/63; G16H 40/20; G16H 40/67

(52) U.S. Cl. CPC G09B 5/06; G06T 11/60; G16H 40/20; G16H 40/67; G06T 2200/24; G06V 40/174; G10L 15/1815; G10L 15/30; G10L 25/63

(57) ABSTRACT

An intelligent medical communication interaction training system based on virtual characters and a method thereof are provided. In the system, a trainee device logs in a communication interaction training and analysis server and selects a training scenario and a virtual character, and provides trainee information or trainee speech and trainee image to the communication interaction training and analysis server, which uses multiple technologies to perform a feature and type analysis for expression, emotion and information of multimodal data on the trainee information or the trainee speech and trainee image to obtain a feature analysis result and type analysis result, which are then processed to obtain a communicate interaction comprehension score and feature, and uses AI to generate and transmit a text response, a speech response, and a facial expression response for the virtual human character data to the trainee device for display and broadcast.

### CROSS-REFERRENCE STATEMENT

This patent application is based on, and claim priority from TAIWAN patent application serial number 114106119, filed on Feb. 19, 2025, the disclosure of which is hereby incorporated by reference herein in its' entirety.

### BACKGROUND OF THE INVENTION

### 1. Field of the Invention

The present invention relates to a medical communication interaction training system and a method thereof, and more particularly to an intelligent medical communication interaction training system based on virtual characters and a method thereof.

### 2. Description of the Related Art

While nation society is entering an aging society and the average life expectancy of national continuously increasing, issues related to long-term care and death trigger medical, economic, and caregiving manpower problems. Therefore, respecting patients' medical autonomy and avoiding unnecessary medical suffering and waste of medical resources, communicating between medical staff and patients based on respect for autonomy, maintaining professionalism, positive and proactive communication to assist patients are important. Furthermore, medical staff also need to learn empathy through professional training and the experience of teachers during the teaching and learning process.

Human emotions and reactions are very complex and varied. Besides the surface meaning, there are hidden meanings in human emotions and reactions. For example, different people may express completely different emotions and reactions when saying “I'm fine.”, for example, the person who says “I'm fine.” could have genuine calmness or hide sadness. However, each person's experience in feeling emotions varies, making it easy or difficult to empathize with others. The existing medical education lacks empathy teaching and training and it is not convenient to perform the training with direct interaction with patient. Conventional medical communication interaction training has certain difficulties and tends to be subjective in evaluation, so artificial intelligence is expected to assist in empathy training to improve the empathy skills of medical staff.

According to above-mentioned contents, what is needed is to develop an improved solution to solve the problem that the conventional medical stall teaching methods lack auxiliary medical communication interaction training.

### SUMMARY OF THE INVENTION

An objective of the present invention is to disclose an intelligent medical communication interaction training system based on virtual characters and a method thereof, to solve the problem that the conventional medical stall teaching methods lack auxiliary medical communication interaction training.

To achieve the objective, the present invention discloses an intelligent medical communication interaction training system based on virtual characters, and the system include a trainee device and a communication interaction training and analysis server, the trainee device includes a first non-transitory computer-readable storage medium and a first processor, and the communication interaction training and analysis server includes a second non-transitory computer-readable storage medium and a second processor.

The first non-transitory computer-readable storage medium is configured to store first computer readable instructions. The first processor is electrically connected to the first non-transitory computer-readable storage medium, and configured to execute the first computer readable instructions to make the trainee device execute operations of: using personally identifiable data to log in a communication interaction training and analysis server to display a training user interface; selecting one of the training scenarios on the training user interface to provide training scenario identification information corresponding to the selected training scenario to the communication interaction training and analysis server; selecting one of at least one virtual character on the training user interface to provide virtual trainee character identification information corresponding to the selected virtual character to the communication interaction training and analysis server; receiving trainee information or a trainee speech, and a trainee image corresponding to the selected virtual character, and providing the selected trainee information or the trainee speech, and the trainee image to the communication interaction training and analysis server; receiving, displaying and playing a text response, a speech response, and a facial expression response corresponding to an unselected one of the at least one virtual character from the communication interaction training and analysis server.

The second non-transitory computer-readable storage medium is configured to store second computer readable instructions and pieces of virtual scenario data, each of the pieces of the virtual scenario data comprises virtual human character data. The second processor is electrically connected to the second non-transitory computer-readable storage medium, and configured to execute the second computer readable instructions to make the trainee device execute operations of: after the trainee device uses the personally identifiable data to log in the communication interaction training and analysis server, providing the training user interface to the trainee device; obtaining the training scenario identification information and the virtual trainee character identification information from the trainee device; querying the virtual human character data corresponding to the training scenario identification information, wherein the queried virtual human character data excludes the virtual human character data corresponding to the virtual trainee character identification information; obtaining the trainee information or the trainee speech, and the trainee image from the trainee device; using a speech-to-text technology, a voice recognition technology, a natural language processing technology, a facial expression recognition technology, and an emotion recognition or sentiment analysis technology to perform an feature analysis and an type analysis for expression, emotion and information of multimodal data on the trainee information or the trainee speech and the trainee image, to obtain a feature analysis result, and a type analysis result, respectively; performing an interaction mode analysis on the feature analysis result and the type analysis result to obtain a communicate interaction comprehension score and a communicate interaction comprehension feature; based on the communicate interaction comprehension score and the communicate interaction comprehension feature, using a generative artificial intelligence to generate the text response, the speech response, and the facial expression response for the virtual human character data; providing the text response, the speech response, and the facial expression response to the trainee device.

Furthermore, the present invention discloses an intelligent medical communication interaction training method based on virtual characters, includes steps of: using personally identifiable data to log in a communication interaction training and analysis server by a trainee device, and providing a training user interface to the trainee device for display, by the communication interaction training and analysis server; selecting one of the training scenarios on the training user interface, and providing training scenario identification information corresponding to the selected training scenario to the communication interaction training and analysis server, by the trainee device; selecting one of the at least one virtual character on the training user interface, and providing virtual trainee character identification information corresponding to the selected virtual character to the communication interaction training and analysis server, by the trainee device; storing pieces of virtual scenario data in the communication interaction training and analysis server, wherein each of the pieces of virtual scenario data comprises virtual human character data; querying the virtual human character data based on the training scenario identification information, by the communication interaction training and analysis server, wherein the queried virtual human character data excludes the virtual human character data corresponding to the virtual trainee character identification information; receiving trainee information or a trainee speech, and a trainee image corresponding to the selected virtual character, and providing the received trainee information or the trainee speech, and the trainee image to the communication interaction training and analysis server, by the trainee device; using a speech-to-text technology, a voice recognition technology, a natural language processing technology, a facial expression recognition technology, and an emotion recognition technology to perform an feature analysis and a type analysis for expression, emotion and information of multimodal data on the trainee information or the trainee speech and the trainee image, to obtain a feature analysis result and a type analysis result, respectively, by the communication interaction training and analysis server; performing an interaction mode analysis on the feature analysis result and the type analysis result to obtain a communicate interaction comprehension score and a communicate interaction comprehension feature, by the communication interaction training and analysis server; based on the communicate interaction comprehension score and the communicate interaction comprehension feature, using a generative artificial intelligence to generate the text response, speech response, and facial expression response corresponding to the queried virtual human character data, respectively, by the communication interaction training and analysis server; providing a text response, a speech response and a facial expression response to the trainee, by the communication interaction training and analysis server; displaying and playing the text response, speech response, and facial expression response corresponding to the unselected one of the at least one virtual character, by the trainee device.

According to the above-mentioned system and method of the present invention, the trainee device logs in the communication interaction training and analysis server by using the personally identifiable data, and selects the training scenario and the virtual character through the training user interface, the trainee device then provides the trainee information or the trainee speech and the trainee image to the communication interaction training and analysis server, the communication interaction training and analysis server uses the speech-to-text technology, the voice recognition technology, the natural language processing technology, the facial expression recognition technology and the emotion recognition to perform the feature analysis and the type analysis for expression, emotion and information of multimodal data on the trainee information or the trainee speech and trainee image to obtain the feature analysis result and the type analysis result, performs the interaction mode analysis based on the feature analysis result and the type analysis result to obtain the communicate interaction comprehension score and the communicate interaction comprehension feature, and uses the generative artificial intelligence to generate the text response, the speech response, and the facial expression response corresponding to the virtual human character data based on the communicate interaction comprehension score and the communicate interaction comprehension feature, and transmit the text response, the speech response, and the facial expression response to the trainee device for display and broadcast, so that a medical communication interaction training can be performed for the trainee in the selected training scenario.

According to the above-mentioned solution, the present invention can achieve the technical effect of intelligent medical communication interaction training based on virtual characters.

### BRIEF DESCRIPTION OF THE DRAWINGS

The structure, operating principle and effects of the present invention will be described in detail by way of various embodiments which are illustrated in the accompanying drawings.

FIG. 1 is a block diagram of an intelligent medical communication interaction training system based on virtual characters, according to the present invention.

FIG. 2 is a schematic view of a training user interface of intelligent medical communication interaction training based on virtual characters, according to the present invention.

FIG. 3 is a schematic view showing a training scenario and virtual characters for intelligent medical communication interaction training based on virtual character, according to the present invention.

FIGS. 4A to 4C are flowcharts of intelligent medical communication interaction training method based on virtual characters, according to the present invention.

### DETAILED DESCRIPTION OF THE PREFERRED EMBODIMENTS

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims.

These embodiments are provided so that this disclosure is thorough and complete, and fully conveys the inventive concept to those skilled in the art. Regarding the drawings, the relative proportions, and ratios of elements in the drawings may be exaggerated or diminished in size for the sake of clarity and convenience. Such arbitrary proportions are only illustrative and not limiting in any way. The same reference numbers are used in the drawings and description to refer to the same or like parts. As used herein, the singular forms “a”, “an” and “the” are intended to include the plural forms as well, unless the context clearly indicates otherwise.

It is to be acknowledged that, although the terms ‘first,’ ‘second,’ ‘third,’ and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the words “comprise” and “include,” and variations such as “comprises,” “comprising,” “includes,” or “including,” will be acknowledged to imply the inclusion of stated elements but not the exclusion of any other elements.

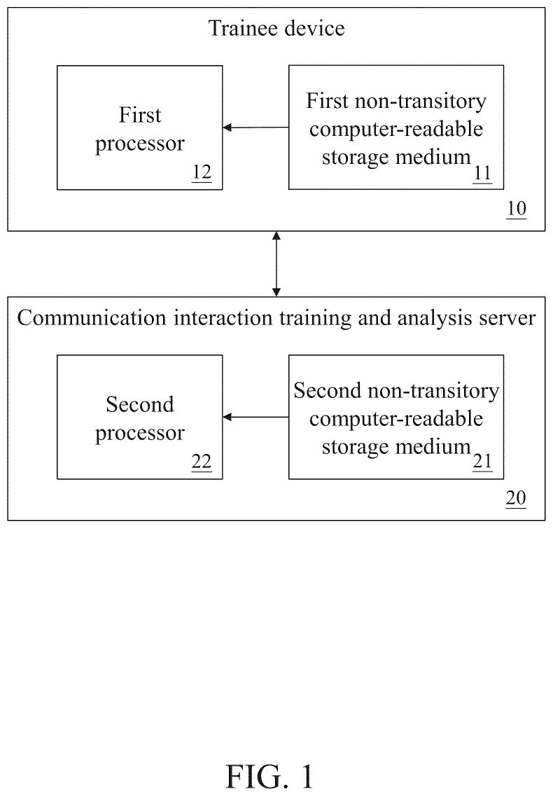

The intelligent medical communication interaction training system based on virtual characters of the present invention will be illustrated in the following paragraphs. Please refer to FIG. 1. FIG. 1 is a block diagram of an intelligent medical communication interaction training system based on virtual characters, according to the present invention.

As shown in FIG. 1, the intelligent medical communication interaction training system includes a trainee device 10 and a communication interaction training and analysis server 20, the trainee device 10 includes a first non-transitory computer-readable storage medium 11 and a first processor 12, the communication interaction training and analysis server 20 includes a second non-transitory computer-readable storage medium 21 and a second processor 22. The trainee device 10 can be, for example, a general computer, laptop, tablet, or smartphone; however, these examples are merely for exemplary illustration, and the application field of the present invention is not limited to these examples.

The first non-transitory computer-readable storage medium 11 stores first computer readable instructions. The first processor 12 is electrically connected to the first non-transitory computer-readable storage medium 11, and configured to the first computer readable instructions to make the trainee device 10 execute the following operations.

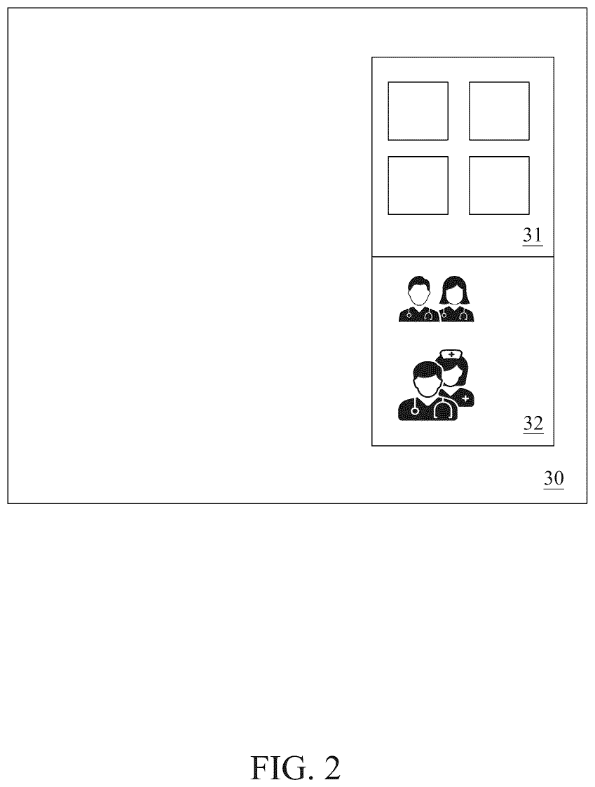

Personally identifiable data is used to log in to a communication interaction training and analysis server 20 to display a training user interface 30. The personally identifiable data can be, for example, a combination of an account and a password, or digital identity certificate, as shown in FIG. 2, which is a schematic view of the training user interface 30. FIG. 2 is a schematic view of a training user interface of an intelligent medical communication interaction training based on virtual characters, according to the present invention. However, these examples are merely for exemplary illustration, and the application field of the present invention is not limited to these examples.

A trainee can operate the trainee device 10 by clicking a mouse or touching, to select one of the training scenarios provided in a training scene selection block 31 of the training user interface 30. However, these examples are merely for exemplary illustration and the application field of the present invention is not limited to these examples. The trainee device 10 provides the training scenario identification information corresponding to the selected training scenario to the communication interaction training and analysis server 20. The training scenario can be, for example, a clinic scenario, medical teaching scenario, nursing scenario, etc., these examples are merely for exemplary illustration, and the application field of the present invention is not limited to these examples.

Next, the trainee operates the trainee device 10 by clicking a mouse or touching, to select one of the at least one virtual character provided in a virtual character selection block 32 of the training user interface 30, the trainee device 10 provides the virtual trainee character identification information corresponding to the selected virtual character to the communication interaction training and analysis server 20. In an embodiment, when the selected training scenario is a clinic scenario, the training user interface 30 can provide a doctor virtual character, a nurse virtual character, and a patient virtual character for the trainee to select; when the selected training scenario is a medical teaching scenario, the training user interface 30 can provide a medical student virtual character and a professor virtual character for the trainee to select; however, these examples are merely for exemplary illustration, and the application field of the present invention is not limited to these examples.

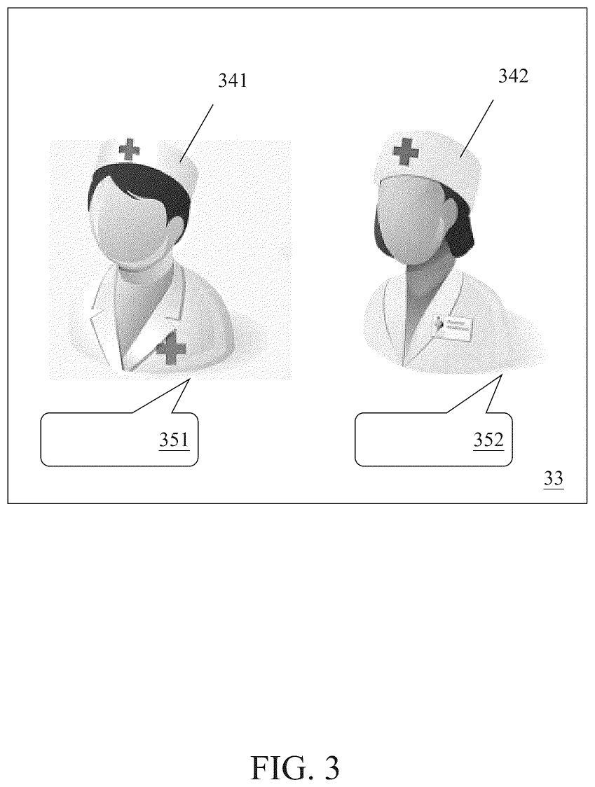

Next, the trainee device 10 can provide the selected training scenario displayed in a training scenario display block 33 of the training user interface 30, display a selected virtual character 341 and an unselected virtual character 342 in the selected training scenario, as shown in FIG. 3, which is a schematic view of the training scenario 33, the selected virtual character 341, and the unselected virtual character 342. FIG. 3 is a schematic view showing a training scenario and a virtual character for intelligent medical communication interaction training based on virtual characters, according to the present invention.

Next, the trainee operates the trainee device 10 by using a keyboard (for example, the keyboard can be a physical keyboard or a virtual keyboard, but these examples are merely for exemplary illustration, and the application field of the present invention is not limited to these examples) to input trainee information of text modality in a text input area 351; alternatively, the trainee can also input a trainee speech of speech modality through a microphone embedded or externally connected to the trainee device 10, the trainee can use an external webcam connected to the trainee device 10 or the built-in camera of a smart device (i.e., the trainee device 10) to capture the trainee image of the image modality. The text input area 351 is displayed adjacent to the selected virtual character 351, the trainee device 10 can provide the trainee information or the trainee speech and the trainee image to the communication interaction training and analysis server 20, the trainee information or the trainee speech and the trainee image correspond to the selected virtual character 351, so that the selected virtual character 351 in the training user interface 30 can instantly present the trainee information and the trainee image of the trainee.

The trainee device 10 receives a text response, a speech response, and a facial expression response corresponding to the unselected virtual character from the communication interaction training and analysis server 20. The trainee device 10 can present the corresponding text response in the text response area 352 corresponding to the unselected virtual character in the training user interface 30, the trainee device 10 can present the facial expression of the virtual character on the unselected virtual character 342 corresponding to the facial expression response in the training user interface 30. The trainee device 10 can play the speech response corresponding to the unselected virtual character in the training user interface 30, so that the trainee can operate the trainee device 10 to provide feedback for the trainee information or the trainee speech and the trainee image during a training period based on the text response, speech response, and facial expression response corresponding to the virtual character, thereby performing the medical communication interaction training for the trainee in the selected training scenario.

It is worth noting that, with the rapid development of virtual character technology, an artificial intelligence can be used to map the selected the corresponding character expressions for the facial expression response of the virtual character to a simple virtual character, and the deepfake technology can also be used to modify and adjust the trainee image or a licensed actual human face image based on the facial expression response of the virtual character, and then overlay the modified and adjusted trainee image or the licensed actual human face image on the virtual character, to make the virtual character present the corresponding facial expression response; however, these examples are merely for exemplary illustration. The technology of displaying the virtual character can refer to existing technology, so detailed description is omitted.

The second non-transitory computer-readable storage medium 21 stores second computer readable instructions, and configured to store pieces of virtual scenario data, each of the pieces of virtual scenario data includes virtual human character data. The second processor 22 is electrically connected to the second non-transitory computer-readable storage medium 21, and configured to execute the second computer readable instructions to make the trainee device 10 execute the following operations.

After the trainee device 10 uses the personally identifiable data to log in the communication interaction training and analysis server 20, the communication interaction training and analysis server 20 provides the training user interface 30 to the trainee device 10, the communication interaction training and analysis server 20 obtains the training scenario identification information and the virtual trainee character identification information from the trainee device 10, and query the corresponding virtual human character data from the second non-transitory computer-readable storage medium 21 based on the training scenario identification information, and the queried virtual human character data excludes the at least one virtual human character data corresponding to the virtual trainee character identification information.

Specifically, in a condition that the training scenario identification information is an identification number being “C101” corresponding to the clinic scenario and the virtual trainee character identification information is identification information being “DR” corresponding to a virtual character of doctor, the training scenario identification information is “C101” with the virtual human character data of a doctor, a nurse, and a patient, the communication interaction training and analysis server 20 queries the virtual human character data corresponding to the doctor, nurse, and patient based on the training scenario identification information being “C101” from the second non-transitory computer-readable storage medium 21, and then excludes the virtual human character data of doctor corresponding to the virtual trainee character identification information of DR, to obtain the virtual human character data corresponding to nurse and patient; however, these examples are merely for exemplary illustration, and the application field of the present invention is not limited to these examples. It is worth noting that, besides the above-mentioned “C101”, the training scenario identification information can also be, for example, “Clinic_205”, or “Examination Room”; besides the above-mentioned “DR”, the virtual trainee character identification information can be “RN” or “PT”, but these examples are merely for exemplary illustration, and the application field of the present invention is not limited to these examples.

After the communication interaction training and analysis server 20 obtains the trainee information or the trainee speech and the trainee image from the trainee device 10, the communication interaction training and analysis server 20 uses a speech-to-text technology, a voice recognition technology, a natural language processing technology, a facial expression recognition technology, and an emotion recognition (also called sentiment analysis) technology to perform an feature analysis and a type analysis for expression, emotion and information of multimodal data on the trainee information or the trainee speech and the trainee image to obtain a feature analysis result and a type analysis result, respectively.

The communication interaction training and analysis server 20 uses the speech-to-text technology and the voice recognition technology to convert the trainee speech of speech modality into the trainee information of text modality, the speech-to-text technology uses an acoustic model and a language model to convert a speech signal into a text, the trainee speech is converted into phonemes, which is the smallest unit of sound in a language, by the acoustic model; the language model is used to predict the word sequence of the phonemes based on context and grammatical rules to improve the accuracy of the converted text. The voice recognition technology analyzes voiceprint features of the trainee speech, the above-mentioned voiceprint features can be, for example, pitch, intensity, speech rate, or formant, but these examples are merely for exemplary illustration, and the application field of the present invention is not limited to these examples. The speech-to-text technology and the voice recognition technology are used to convert the trainee speech of speech modality into the trainee information of text modality.

The communication interaction training and analysis server 20 uses the facial expression recognition technology to perform facial expression analysis on the trainee image to obtain the facial expression of the trainee. The facial expression recognition technology is based on an artificial intelligence technology and a computer vision technology. The communication interaction training and analysis server 20 performs facial detection algorithms (such as Haar feature classifier, deep learning model, etc.) to locate a face area of the trainee in the conversation video, then, marks key parts (such as the corners of the eyes, corners of the mouth, eyebrows, etc.) of the face area of the trainee, and extracts geometric or appearance features redisplaying the facial expression from these key parts. Based on the face area of the trainee, the marked key parts, and the geometric or appearance features of the facial expression, a machine learning model or a deep learning model can be used to perform mapping of predefined facial expression types (such as happiness, sadness, surprise, anger, disgust, fear, etc.).

The communication interaction training and analysis server 20 uses an emotion recognition (also called sentiment analysis) technology to perform an emotion analysis on the trainee information or the trainee speech and the trainee image, to obtain the emotion of the trainee. The emotion recognition technology is an artificial intelligence technology that provides recognition and analysis of individual emotions by processing data such as voice, text or facial expressions to infer the emotional response of the individual. The communication interaction training and analysis server 20 uses a natural language processing technology on the trainee information, to extract emotional words and sentences to infer the emotion of the trainee based on vocabulary and grammar; the communication interaction training and analysis server 20 can infer the emotion of the trainee based on the voiceprint feature of the trainee speech, for example, when the voiceprint feature is a high-pitched tone, it infers that the emotion of the trainee is excitement; when the voiceprint feature is a low-pitched tone, it infers that the emotion of the trainee is sadness; however, these examples are merely for exemplary illustration, and the application field of the present invention is not limited to these examples. The communication interaction training and analysis server 20 calls the obtained facial expression of the trainee and combines it with the inferred emotion of the trainee for comprehensive analysis to obtain the emotion of the trainee.

The communication interaction training and analysis server 20 uses the natural language processing technology to perform semantic and lexical analysis on the trainee information to obtain an information analysis result, the communication interaction training and analysis server 20 uses the above-mentioned speech-to-text technology, voice recognition technology, natural language processing technology, facial expression recognition technology, and emotion recognition technology to perform the feature analysis and the type analysis on the expression, emotion and information of multimodal data on the trainee information or the trainee speech and the trainee image, to obtain the feature analysis result and the type analysis result.

Next, the communication interaction training and analysis server 20 performs an interaction mode analysis on the feature analysis result and the type analysis result to obtain a communicate interaction comprehension score and a communicate interaction comprehension feature. It is worth noting that the communication interaction training and analysis server 20 can use an empathy analysis model to perform an interaction mode analysis on the feature analysis result and the type analysis result to obtain the communicate interaction comprehension score and the communicate interaction comprehension feature, in an embodiment, the empathy analysis model can apply the natural language processing technology, artificial neural networks (ANNs), long short-term memory networks (LSTM), or transformer models (however, these examples are merely for exemplary illustration, and the application field of the present invention is not limited to these examples) to perform emotion analysis, semantic understanding, emotional simulation, and understanding of empathetic responses,4 to analyze the interaction mode of the training, and then obtain the communicate interaction comprehension score and the communicate interaction comprehension feature based on the interaction mode analysis.

Based on the communicate interaction comprehension score and the communicate interaction comprehension feature, the communication interaction training and analysis server 20 can use a generative artificial intelligence to generate the text response, speech response, and facial expression response corresponding to the virtual human character data. In an embodiment, in the clinic scenario, based on the communicate interaction comprehension score of “40” and the communicate interaction comprehension feature of “indifference”, the communication interaction training and analysis server 20 uses the generative artificial intelligence to generate the corresponding text response for the virtual human character data of the nurse as “the reply content is too indifferent, please adjust the reply content and retrain”, the corresponding speech response of speech modality as “the reply content is too indifferent, please adjust the reply content and retrain” and the corresponding facial expression response as “shock”; based on the communicate interaction comprehension score of “40” and the communicate interaction comprehension feature of “indifference”, the communication interaction training and analysis server 20 can use the generative artificial intelligence to generate the corresponding text response of text modality for the virtual human character data of the nurse as “I feel the doctor's reply is a bit indifferent”, the speech response of speech modality as “I feel the doctor's reply is a bit indifferent” and the corresponding facial expression response as “surprise”. However, these examples are merely for exemplary illustration, and the application field of the present invention is not limited to these examples. The communication interaction training and analysis server 20 provides the text response, the speech response, and the facial expression response to the trainee device 10.

It is to be particularly noted that, in actual implementation, the above-mentioned solution of the present invention can be implemented fully or partly based on hardware, for example, one or more component of the system can be implemented by hardware processor, such as integrated circuit chip, system on chip (SoC), a complex programmable logic device (CPLD), or a field programmable gate array (FPGA). The non-transitory computer-readable storage medium records computer readable program instructions, and the processor can execute the computer readable program instructions to implement concepts of the present invention. The non-transitory computer-readable storage medium can be a tangible apparatus for holding and storing the instructions executable of an instruction executing apparatus. The non-transitory computer-readable storage medium can be, but not limited to electronic storage apparatus, magnetic storage apparatus, optical storage apparatus, electromagnetic storage apparatus, semiconductor storage apparatus, or any appropriate combination thereof. More particularly, the non-transitory computer-readable storage medium can include a hard disk, an RAM memory, a read-only-memory, a flash memory, an optical disk, a floppy disc, or any appropriate combination thereof, but this exemplary list is not an exhaustive list. The non-transitory computer-readable storage medium is not interpreted as the instantaneous signal such a radio wave or other freely propagating electromagnetic wave, or electromagnetic wave propagated through waveguide, or other transmission medium (such as optical signal transmitted through fiber cable), or electric signal transmitted through electric wire. Furthermore, the computer readable program instruction can be downloaded from the non-transitory computer-readable storage medium to each calculating/processing apparatus, or downloaded through network, such as internet network, local area network, wide area network and/or wireless network, to external computer equipment or external storage apparatus. The network includes copper transmission cable, fiber transmission, wireless transmission, router, firewall, switch, hub and/or gateway. The network card or network interface of each calculating/processing apparatus can receive the computer readable program instructions from network, and forward the computer readable program instruction to store in non-transitory computer-readable storage medium of each calculating/processing apparatus. The computer readable instructions can be executed by the server 120. The computer readable instructions for executing the operations of the present invention can be assembly language instructions, instruction-set-structure instructions, machine instructions, machine-related Instructions, micro-instructions, firmware instructions, or source codes or object codes written in any combination of one or more programming languages. The programming language includes object-oriented programming languages, such as: Common Lisp, Python, C++, Objective-C, Smalltalk, Delphi, Java, Swift, C#, Perl, Ruby, or PHP; the programming language can include regular procedural programming languages, such as C language or similar programming languages.

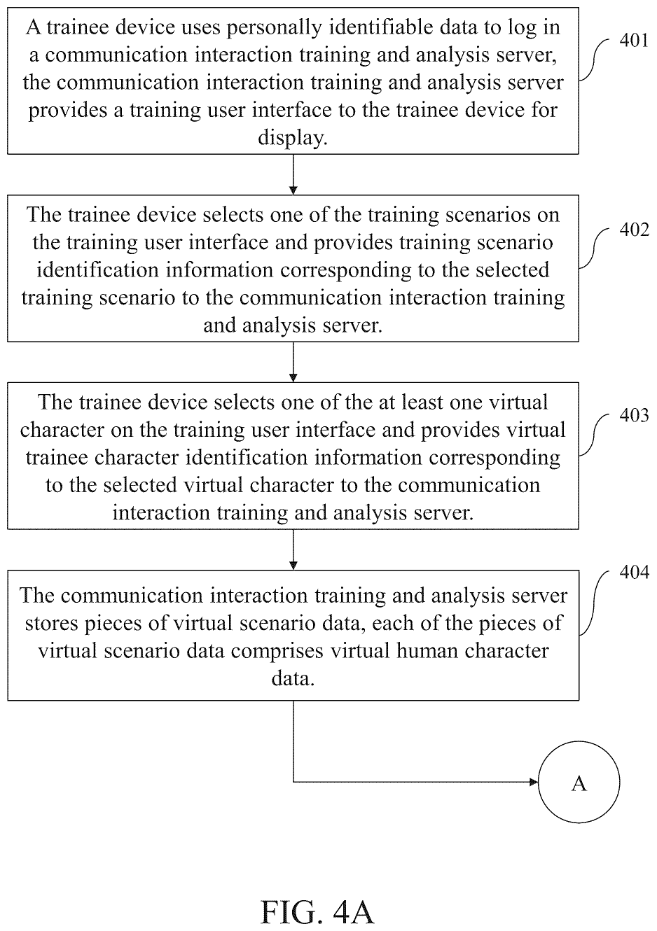

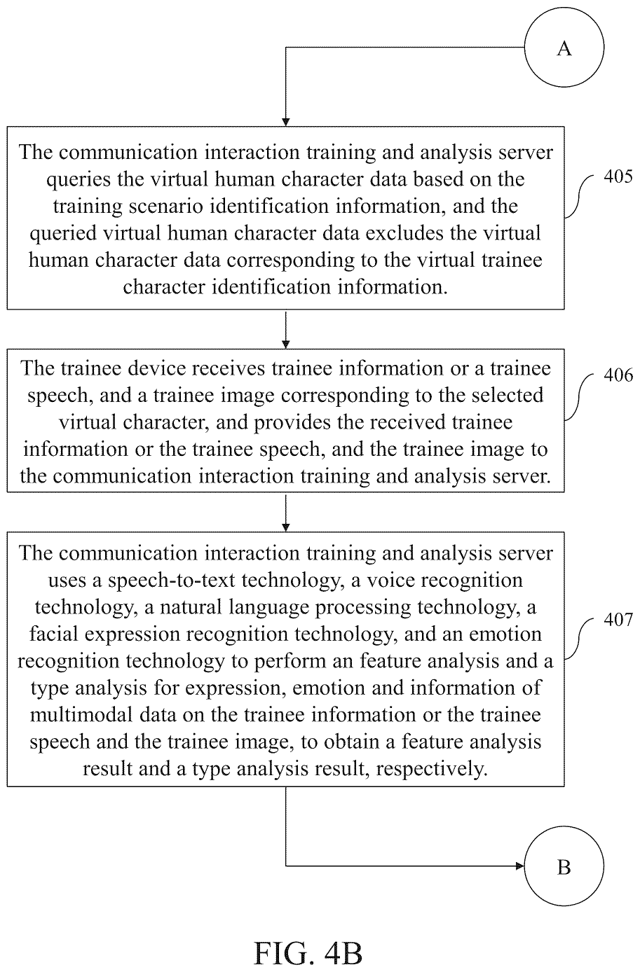

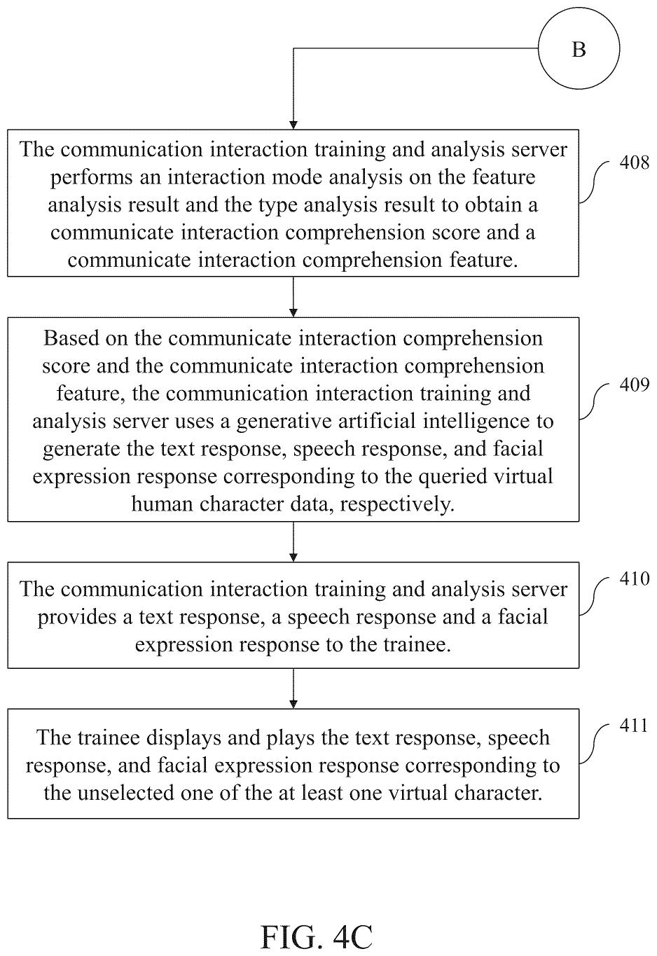

An operation of the method of the present invention will be illustrated in the following paragraphs. Please refer to FIG. 4A to FIG. 4C, which are flowcharts of an intelligent medical communication interaction training method based on virtual characters, according to the present invention.

As shown in FIG. 4A to FIG. 4C, the intelligent medical communication interaction training method disclosed in the present invention, includes the following steps.

In a step 401, a trainee device uses personally identifiable data to log in a communication interaction training and analysis server, the communication interaction training and analysis server provides a training user interface to the trainee device for display. In a step 402, the trainee device selects one of the training scenarios on the training user interface and provides training scenario identification information corresponding to the selected training scenario to the communication interaction training and analysis server. In a step 403, the trainee device selects one of the at least one virtual character on the training user interface and provides virtual trainee character identification information corresponding to the selected virtual character to the communication interaction training and analysis server. In a step 404, the communication interaction training and analysis server stores pieces of virtual scenario data, each of the pieces of virtual scenario data comprises virtual human character data. In a step 405, the communication interaction training and analysis server queries the virtual human character data based on the training scenario identification information, and the queried virtual human character data excludes the virtual human character data corresponding to the virtual trainee character identification information. In a step 406, the trainee device receives trainee information or a trainee speech, and a trainee image corresponding to the selected virtual character, and provides the received trainee information or the trainee speech, and the trainee image to the communication interaction training and analysis server. In a step 407, the communication interaction training and analysis server uses a speech-to-text technology, a voice recognition technology, a natural language processing technology, a facial expression recognition technology, and an emotion recognition technology to perform an feature analysis and a type analysis for expression, emotion and information of multimodal data on the trainee information or the trainee speech and the trainee image, to obtain a feature analysis result and a type analysis result, respectively. In a step 408, the communication interaction training and analysis server performs an interaction mode analysis on the feature analysis result and the type analysis result to obtain a communicate interaction comprehension score and a communicate interaction comprehension feature. In a step 409, based on the communicate interaction comprehension score and the communicate interaction comprehension feature, the communication interaction training and analysis server uses a generative artificial intelligence to generate the text response, speech response, and facial expression response corresponding to the queried virtual human character data, respectively. In a step 410, the communication interaction training and analysis server provides a text response, a speech response and a facial expression response to the trainee. In a step 411, the trainee displays and plays the text response, speech response, and facial expression response corresponding to the unselected one of the at least one virtual character.

According to above-mentioned contents, the trainee device logs in the communication interaction training and analysis server by using the personally identifiable data, and selects the training scenario and the virtual character through the training user interface, the trainee device then provides the trainee information or the trainee speech and the trainee image to the communication interaction training and analysis server, the communication interaction training and analysis server uses the speech-to-text technology, the voice recognition technology, the natural language processing technology, the facial expression recognition technology and the emotion recognition to perform the feature analysis and the type analysis for expression, emotion and information of multimodal data on the trainee information or the trainee speech and trainee image to obtain the feature analysis result and the type analysis result, performs the interaction mode analysis based on the feature analysis result and the type analysis result to obtain the communicate interaction comprehension score and the communicate interaction comprehension feature, and uses the generative artificial intelligence to generate the text response, the speech response, and the facial expression response corresponding to the virtual human character data based on the communicate interaction comprehension score and the communicate interaction comprehension feature, and transmit the text response, the speech response, and the facial expression response to the trainee device for display and broadcast, so that a medical communication interaction training can be performed for the trainee in the selected training scenario.

According to the above-mentioned solution, the present invention can solve the problem that the conventional medical stall teaching methods lack auxiliary medical communication interaction training, to achieve the technical effect of improving intelligent medical communication interaction training based on virtual characters.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. An intelligent medical communication interaction training system based on virtual characters, comprising:
a trainee device, comprising:
a first non-transitory computer-readable storage medium, configured to store first computer readable instructions; and
a first processor, electrically connected to the first non-transitory computer-readable storage medium, and configured to execute the first computer readable instructions to make the trainee device execute:
using personally identifiable data to log in a communication interaction training and analysis server to display a training user interface;
selecting one of the training scenarios on the training user interface to provide training scenario identification information corresponding to the selected training scenario to the communication interaction training and analysis server;
selecting one of at least one virtual character on the training user interface to provide virtual trainee character identification information corresponding to the selected virtual character to the communication interaction training and analysis server;
receiving trainee information or a trainee speech, and a trainee image corresponding to the selected virtual character, and providing the selected trainee information or the trainee speech, and the trainee image to the communication interaction training and analysis server; and
receiving, displaying and playing a text response, a speech response, and a facial expression response corresponding to an unselected one of the at least one virtual character from the communication interaction training and analysis server; and
the communication interaction training and analysis server comprising:
a second non-transitory computer-readable storage medium, configured to store second computer readable instructions and pieces of virtual scenario data, each of the pieces of the virtual scenario data comprises virtual human character data; and
a second processor, electrically connected to the second non-transitory computer-readable storage medium, and configured to execute the second computer readable instructions to make the trainee device execute:
after the trainee device uses the personally identifiable data to log in the communication interaction training and analysis server, providing the training user interface to the trainee device;
obtaining the training scenario identification information and the virtual trainee character identification information from the trainee device;
querying the virtual human character data corresponding to the training scenario identification information, wherein the queried virtual human character data excludes the virtual human character data corresponding to the virtual trainee character identification information;
obtaining the trainee information or the trainee speech, and the trainee image from the trainee device;
using a speech-to-text technology, a voice recognition technology, a natural language processing technology, a facial expression recognition technology, and an emotion recognition or sentiment analysis technology to perform an feature analysis and an type analysis for expression, emotion and information of multimodal data on the trainee information or the trainee speech and the trainee image, to obtain a feature analysis result, and a type analysis result, respectively;
performing an interaction mode analysis on the feature analysis result and the type analysis result to obtain a communicate interaction comprehension score and a communicate interaction comprehension feature;
based on the communicate interaction comprehension score and the communicate interaction comprehension feature, using a generative artificial intelligence to generate the text response, the speech response, and the facial expression response for the virtual human character data; and
providing the text response, the speech response, and the facial expression response to the trainee device.

2. The intelligent medical communication interaction training system based on virtual characters according to claim 1, wherein the communication interaction training and analysis server provides different numbers of the at least one virtual character based on the selected training scenario.

3. The intelligent medical communication interaction training system based on virtual characters according to claim 1, wherein the communication interaction training and analysis server uses artificial intelligence to select a character expression corresponding to the facial expression response of the virtual character and map the selected character expressions to the virtual character.

4. The intelligent medical communication interaction training system based on virtual characters according to claim 1, wherein the communication interaction training and analysis server uses a deepfake technology to modify and adjust the trainee image or licensed actual human face image based on the facial expression response of the virtual character, and overlay the modified and adjusted trainee image or the licensed actual human face image on the virtual character.

5. An intelligent medical communication interaction training method based on virtual characters, comprising:
using personally identifiable data to log in a communication interaction training and analysis server by a trainee device, and providing a training user interface to the trainee device for display, by the communication interaction training and analysis server;
selecting one of the training scenarios on the training user interface, and providing training scenario identification information corresponding to the selected training scenario to the communication interaction training and analysis server, by the trainee device;
selecting one of the at least one virtual character on the training user interface, and providing virtual trainee character identification information corresponding to the selected virtual character to the communication interaction training and analysis server, by the trainee device;
storing pieces of virtual scenario data in the communication interaction training and analysis server, wherein each of the pieces of virtual scenario data comprises virtual human character data;
querying the virtual human character data based on the training scenario identification information, by the communication interaction training and analysis server, wherein the queried virtual human character data excludes the virtual human character data corresponding to the virtual trainee character identification information;
receiving trainee information or a trainee speech, and a trainee image corresponding to the selected virtual character, and providing the received trainee information or the trainee speech, and the trainee image to the communication interaction training and analysis server, by the trainee device;
using a speech-to-text technology, a voice recognition technology, a natural language processing technology, a facial expression recognition technology, and an emotion recognition technology to perform an feature analysis and a type analysis for expression, emotion and information of multimodal data on the trainee information or the trainee speech and the trainee image, to obtain a feature analysis result and a type analysis result, respectively, by the communication interaction training and analysis server;
performing an interaction mode analysis on the feature analysis result and the type analysis result to obtain a communicate interaction comprehension score and a communicate interaction comprehension feature, by the communication interaction training and analysis server;
based on the communicate interaction comprehension score and the communicate interaction comprehension feature, using a generative artificial intelligence to generate the text response, speech response, and facial expression response corresponding to the queried virtual human character data, respectively, by the communication interaction training and analysis server;
providing a text response, a speech response and a facial expression response to the trainee, by the communication interaction training and analysis server; and
displaying and playing the text response, speech response, and facial expression response corresponding to the unselected one of the at least one virtual character, by the trainee device.

6. The intelligent medical communication interaction training method based on virtual characters according to claim 5, wherein the communication interaction training and analysis server provides different numbers of the at least one virtual character based on the selected training scenario.

7. The intelligent medical communication interaction training method based on virtual characters according to claim 5, wherein the communication interaction training and analysis server uses artificial intelligence to select a character expression corresponding to the facial expression response of the virtual character and map the selected character expressions to the virtual character.

8. The intelligent medical communication interaction training method based on virtual characters according to claim 5, wherein the communication interaction training and analysis server uses a deepfake technology to modify and adjust the trainee image or licensed actual human face image based on the facial expression response of the virtual character, and overlay the modified and adjusted trainee image or the licensed actual human face image on the virtual character.
